(19) United States
(12) Patent Application Publication
(10) Pub. No.: US 2021/0115748 A9
(43) Pub. Date: Apr. 22, 2021

(54) TOPICAL COMPOSITIONS AND METHODS OF TREATMENT

(71) Applicant: Patagonia Pharmaceuticals LLC, Woodcliff Lake, NJ (US)

(72) Inventor: Zachary Rome, Nyack, NY (US)

(21) Appl. No.: 16/497,726

(22) PCT Filed: Mar. 26, 2018
(86) PCT No.: PCT/US2018/024261

§ 371 Date: Sep. 25, 2019

Related U.S. Application Data
(60) Provisional application No. 62/476,919, filed on Mar. 27, 2017.

(51) Int. Cl. E21B 33/08; E21B 47/08

(52) U.S. Cl. CPC E21B 33/085; E21B 47/06; E21B 47/08

(57) ABSTRACT

The present invention relates to topical compositions containing a therapeutically effective amount of tetrahydrocannabinol. The compositions are useful for treating a patient suffering from conditions such as pain and pruritus, particularly neuropathic or chronic inflammatory pain and/or pruritus.

### FIELD OF THE INVENTION

The present invention relates to topical compositions containing a therapeutically effective amount of tetrahydrocannabinol (also known as THC). The compositions are useful for treating a patient suffering from conditions such as pain and pruritus, particularly neuropathic or chronic inflammatory pain and/or pruritus.

### BACKGROUND OF THE INVENTION

Pain and pruritus represent significant public health issues. According to the National Institutes of Health (NIH), pain affects more Americans than diabetes, heart disease, and cancer combined. Pain is cited as the most common reason Americans access the health care system, and chronic pain is the most common cause of long-term disability. See, https://report.nih.gov/nihfactsheets/ViewFactSheet.aspx?csid=57. Pruritus, also more commonly known as itch, can be similarly debilitating, and it is often cited as the most common symptom in dermatology. Chronic pruritus, i.e. pruritus lasting greater than six weeks, has been found to occur in 16.7% of the general population. See, https://www.ncbi.nlm.nih.gov/pubmed/20924157 and https://www.ncbi.nlm.nih.gov/books/NBK200924/.

Despite the common symptoms of pain and pruritus, the underlying etiology and mechanisms can be complex and very different. Pain can be generally separated into four categories: somatic pain, visceral pain, neuropathic pain, and psychogenic pain. Somatic pain is caused by the activation of pain receptors in either the body surface or musculoskeletal tissues. Visceral pain is the pain derived from damage or injury to the internal organs. Neuropathic pain is caused by injury to or malfunction of the spinal cord and peripheral nerves. Psychogenic pain is physical pain that is caused, increased, or prolonged by mental, emotional, or behavioral factors. Regarding pruritus, Potenzieri and Undem, Clin Exp Allergy. 2012 January; 42(1):8-19 have broadly delineated itch into four subtypes: pruriceptive, neuropathic, neurogenic, and psychogenic. Pruriceptive itch is a sensation of itch that originates following activation of primary afferent nerve terminals. This type of itch is associated with insect bites or intradermal injection of pruritic substances. Neuropathic itch results from nerve injury. Neurogenic itch refers to itch resulting from central nervous system (CNS) activation without necessary activation of sensory nerve fibers. Psychogenic itch results from underlying mental illness. Furthermore, both pain and pruritus can be acute or chronic. Although separate specific pathways for pain and itch processing have been uncovered, recent research has revealed that there are overlapping functions in primary afferents. These common mechanisms appear to be most relevant for conditions of neuropathic or chronic inflammatory pain and/or itch. See, Schmelz, M. (2015). Itch and Pain Differences and Commonalities. Pain Control Handbook of Experimental Pharmacology, 285-301.

Some examples of drugs that have been used to treat pain and/or pruritus include NSAIDS, acetaminophen, antidepressants, anti-seizure medicines, steroids, opioids, analgesics, and antihistamines. These drugs, however, are not always safe, effective, or appropriate and there is still a great unmet need for improved treatments. This unmet need is particularly true for patients suffering from neuropathic pain and pruritus. For example, up to 50% of patients with neuropathic pain do not respond to any treatment, and there are currently no FDA approved treatments for neuropathic pruritus.

Cannabinoids are a class of compounds that act on the cannabinoid receptors in cells. Cannabinoids can be endogenous, wherein they are called endocannabinoids, synthetic, or derived from plants, wherein they are called phytocannabinoids. A well-known source of phytocannabinoids is the genus of plants known as Cannabis or more colloquially referred to as marijuana. At least 104 phytocannabinoids have been isolated from marijuana to date, and many of these compounds have widely variable biological activities and properties.

Marijuana is now legalized for use for medicinal purposes in 28 states and the District of Columbia, and cannabinoids have been reported to provide various therapeutic benefits for humans, including effects that are anti-psychotic, analgesic, anti-inflammatory, anti-spasmodic, anti-convulsant, anti-emetic, antioxidant, neuroprotective, and immunomodulatory. Cannabinoid compounds are generally administered by ingestion, inhalation, or transdermally to achieve systemic distribution of the active compounds for targeting of the central nervous system. Certain cannabinoids and combinations thereof have been investigated for their benefit in treating symptoms of pain; however, there are significant challenges and issues relating to their safe, effective, and federally acceptable use. These issues include, but are not limited to, issues relating to side effects, mechanisms of action, composition, consistency, quality control, product sourcing, and cost.

It is apparent from the foregoing that there is an ongoing need for developing safe and effective treatments for patients suffering from pain and pruritus. Cannabinoids may offer important treatment opportunities, but there are significant issues with the safe, effective, and federally acceptable use of the current cannabinoid treatment options.

It has surprisingly been found in the present invention that the cannabinoid compound, tetrahydrocannabinol, can be safely and effectively administered topically to treat and provide relief for patients from conditions such as pain and pruritus, particularly neuropathic or chronic inflammatory pain and/or pruritus.

### SUMMARY OF THE INVENTION

The present invention relates to topical compositions containing a therapeutically effective amount of tetrahydrocannabinol. The compositions are useful for treating a patient suffering from conditions such as pain and pruritus, particularly neuropathic or chronic inflammatory pain and/or pruritus.

The present invention is based on the surprising discovery that the topical application (i.e. the local delivery) of tetrahydrocannabinol is useful for treating symptoms associated with pain and pruritus, particularly neuropathic or chronic inflammatory pain and/or pruritus. It was further discovered that amelioration of these symptoms can be achieved with significantly lower quantities of tetrahydrocannabinol than previously used or contemplated, and that the inclusion of additional cannabinoid compounds with the tetrahydrocannabinol is not necessary.

Consequently, the compositions provided herein provide a safer, more consistent, more cost effective, and more federally acceptable method of treating pain and pruritus with cannabinoids, and with less side effects.

While it is known that certain cannabinoids and combinations thereof can provide relief from pain, there are many uncertainties regarding the safety, efficacy, and commercial viability of these compounds and/or products. For example, reports are variable and inconsistent with regards to which specific cannabinoids might be useful for which specific types of pain or pruritus and in which type of dosage form, dosage amount, and route of administration, and with which excipients. Furthermore, the mechanisms of action are not completely understood and nuances between these variables continue to produce variable and/or inconsistent results. For example, a 2007 study found that, for patients with neuropathic pain, smoking cannabis provided no acute benefit on pain relief and only a “modest” benefit on average daily pain relief when compared to other drugs such as gabapentin. However, a different study in 2007 found that, in HIV patients with neuropathic pain, smoking cannabis provided significant pain relief that was comparable to the benefit provided from gabapentin. See, https://www.ncbi.nlm.nih.gov/pmc/articles/PMC2950205/ and http://safeaccess.ca/research/pdf/hiv_pain_cannabis_abrams.pdf.

As another example, Cannador®, a cannabis extract administered in oral capsules containing a mixture of tetrahydrocannabinol and cannabidiol (another cannabinoid that is also known as CBD) in a ratio that is generally approximately 2:1, did not demonstrate any benefit in patients with post-herpetic neuralgia. However, Sativex®, a transmucosal spray containing tetrahydrocannabinol and cannabidiol in a 1:1 ratio, demonstrated pain relief in patients with neuropathic pain. See, Ernst G, et al. Standardized cannabis extract in the treatment of postherpetic neuralgia: a randomized, double-blind, placebo-controlled cross-over study; International Association for Cannabis as Medicine; 2005 Sep. 9; Leiden, Netherlands. 2005, and https://www.ncbi.nlm.nih.gov/pubmed/17997224. These seemingly contradictory results might lead one to question whether Cannador's oral route of administration is the cause for the lack of efficacy. However, Cannador has demonstrated efficacy in several other pain conditions, including pain associated with multiple sclerosis and post-operative pain. See, http://www.thelancet.com/journals/lancet/article/PIIS0140673603147381/abstract and https://www.ncbi.nlm.nih.gov/pubmed/16645457.

Cannabidiol is a non-psychoactive phytocannabinoid that can account for up to 40% of a cannabis plant extract. It is considered to have a wide scope of medical applications and has been the subject of substantial research in recent years. This research suggests that cannabidiol could be a crucial component in achieving pain relief when used in combination with other cannabinoids. For example, results from a three-arm, Phase III study (N=177) comparing Sativex, a tetrahydrocannabinol extract, and a placebo, in patients suffering from intractable cancer pain that is unresponsive to opiates, demonstrated that Sativex produced statistically significant improvements in analgesia, while the tetrahydrocannabinol extract failed to produce a statistical demarcation from placebo. These results suggest that the addition of cannabidiol was critical to obtaining significant pain relief. See, Johnson, J. R. and Potts R., Cannabis-based medicines in the treatment of cancer pain: a randomised, double-blind, parallel group, placebo controlled, comparative study of the efficacy, safety and tolerability of Sativex and Tetranabinex in patients with cancer-related pain. Edinburgh, Scotland: 2005. Mar., 8-11.

As another example, Cannador demonstrated a decrease in pain intensity in patients with post-operative pain; whereas dronabinol (a synthetic form of tetrahydrocannabinol in an oral capsule), did not demonstrate any benefit over placebo in patients with post-operative pain, and nabilone (a synthetic dimethyl-heptyl analogue of tetrahydrocannabinol) actually increased pain in a randomly controlled trial in patients with post-operative pain. See, https://www.ncbi.nlm.nih.gov/pubmed/14581124 and https://www.ncbi.nlm.nih.gov/pubmed/16873343. In view of these prior art teachings regarding the apparent criticality of cannabidiol in pain management, it was therefore surprising that we discovered tetrahydrocannabinol alone to be an effective means of pain relief when applied topically.

Furthermore, we have surprisingly found that the compositions disclosed herein are therapeutically effective treatments for pain and pruritus at significantly lower doses than previously used or contemplated. For example, two studies conducted in France on dronabinol for treating chronic neuropathic pain failed to show a significant benefit for pain reduction or other parameters, and showed frequent adverse events requiring discontinuation with doses averaging 15 to 16.6 mg of tetrahydrocannabinol. See, https://www.ncbi.nlm.nih.gov/pubmed/12496714 and https://www.ncbi.nlm.nih.gov/pubmed/14987627. According to the Centers for Disease Control and Prevention, the average weight in the United States for an adult man is 195.5 pounds or 88.7 kilograms and 166.2 pounds or 75.4 kilograms for adult women. Therefore, if one considers the lowest end of the average dosing disclosed in the French study, namely the 15 mg dose, these studies show that an approximate dosing of 0.169 mg/kg in men and 0.199 mg/kg in women did not produce any benefit on pain or other parameters, and yet resulted in frequent adverse events. Also, the currently FDA approved products Marinol (containing dronabinol) and nabilone (a synthetic tetrahydrocannabinol mimic) are dosed at relatively high concentrations, although these products are approved for indications other than pain and pruritus, namely for use an anti-emetic for chemotherapy patients and as an appetite stimulant for AIDS patients. In contrast, the compositions of the present invention have demonstrated effectiveness at topical dosing as low as 0.003 mg/kg, with no evidence of adverse events.

### BRIEF DESCRIPTION OF THE DRAWINGS

FIG. 1 shows the average body weight in the various treatment groups throughout the study (grams), as described in Example 2 from the Complete Freund's Adjuvant (CFA) induced model of monoarthritis in rats.

FIG. 2 shows average body weight gain throughout the study (percent of initial weight), as described in Example 2 from the Complete Freund's Adjuvant (CFA) induced model of monoarthritis in rats.

FIG. 3 shows results for an incapacitance test on Days 14, 16 and 19 of the study for the treatments: vehicle (negative control), morphine (positive control) and three tetrahydrocannabinol concentrations, as described in Example 2 from the Complete Freund's Adjuvant (CFA) induced model of monoarthritis in rats.

FIG. 4 shows results for an incapacitance test on Day 19 of the study for the treatments: vehicle (negative control), morphine (positive control) and the three tetrahydrocannabinol concentrations combined, as described in Example 2 from the Complete Freund's Adjuvant (CFA) induced model of monoarthritis in rats.

FIG. 5 shows results for the von Frey test on Days 14, 16 and 19 of the study for the treatments: vehicle (negative control), morphine (positive control) and three tetrahydrocannabinol concentrations, as described in Example 2 from the Complete Freund's Adjuvant (CFA) induced model of monoarthritis in rats.

### DETAILED DESCRIPTION OF THE INVENTION

The present invention relates to a composition for topical delivery of tetrahydrocannabinol comprising a therapeutically effective amount of tetrahydrocannabinol and a pharmaceutically acceptable carrier.

In another aspect, the present invention relates to a composition wherein the tetrahydrocannabinol has a purity of 95% by weight or greater as determined by HPLC.

In another aspect, the present invention relates to a composition comprising about 0.005% to about 25% by weight tetrahydrocannabinol.

In another aspect, the present invention relates to a composition comprising about 0.01% to about 15% by weight tetrahydrocannabinol.

In another aspect, the present invention relates to a composition comprising about 0.05% to about 10% by weight tetrahydrocannabinol.

In another aspect, the present invention relates to a composition comprising about 0.1% to about 5% by weight tetrahydrocannabinol.

In another aspect, the present invention relates to a composition comprising about 0.25% to about 2.5% by weight tetrahydrocannabinol.

In another aspect, the present invention relates to a composition comprising about 0.1% by weight tetrahydrocannabinol.

In another aspect, the present invention relates to a composition comprising about 1% by weight tetrahydrocannabinol.

In another aspect, the present invention relates to a composition comprising about 2.5% by weight tetrahydrocannabinol.

In another aspect, the present invention relates to a composition comprising about 5% by weight tetrahydrocannabinol.

In another aspect, the present invention relates to a composition for administration to a human patient or animal.

In another aspect, the present invention relates to a composition for administration to a human patient.

In another aspect, the present invention relates to a composition in the form of a unit dosage composition.

In another aspect, the present invention relates to a unit dosage composition comprising from about 0.001 to about 1 mg/kg of tetrahydrocannabinol, based on the weight of the human patient.

In another aspect, the present invention relates to a unit dosage composition comprising from about 0.003 to about 0.5 mg/kg of tetrahydrocannabinol, based on the weight of the human patient.

In another aspect, the present invention relates to a unit dosage composition comprising from about 0.003 mg/kg of tetrahydrocannabinol, based on the weight of the human patient.

In another aspect, the present invention relates to a unit dosage composition comprising about 0.01 mg/kg of tetrahydrocannabinol, based on the weight of the human patient.

In another aspect, the present invention relates to a unit dosage composition comprising about 0.05 mg/kg of tetrahydrocannabinol, based on the weight of the human patient.

In another aspect, the present invention relates to a unit dosage composition comprising about 0.1 mg/kg of tetrahydrocannabinol, based on the weight of the human patient.

In another aspect, the present invention relates to a unit dosage composition comprising about 0.5 mg/kg of tetrahydrocannabinol, based on the weight of the human patient.

In another aspect, the present invention relates to a unit dosage composition demonstrating at least one of the following pharmacokinetic parameters selected from a Cₘₐₓ less than about 1.32 ng/ml or an AUC less than about 2.88 ng hr/ml.

In another aspect, the present invention relates to a composition wherein the pharmaceutically acceptable carrier comprises one or more materials selected from plant-based oils, alcohols, dipropylene glycol, ethyl acetate, ethyl lactate, ethyl oleate, glycerin, isopropyl myristate, isopropyl palmitate, medium-chain triglycerides, mineral oil, petrolatum, silicone oil polyethylene glycol, propylene glycol, tricaprylin, dimethyl isosorbide, water, and mixtures thereof.

In another aspect, the present invention relates to a composition wherein the plant based oil is selected from oils derived from fruits, vegetables, flowers, nuts, or seeds.

In another aspect, the present invention relates to a composition wherein the plant based oil is selected from sesame oil, olive oil, peanut oil, castor oil, almond oil, canola oil, corn oil, cottonseed oil, safflower oil, soybean oil, sunflower oil, and mixtures thereof.

In another aspect, the present invention relates to a composition wherein the alcohol is selected from ethanol, benzyl alcohol, isopropyl alcohol, and mixtures thereof.

In another aspect, the present invention relates to a composition wherein the pharmaceutically acceptable carrier comprises one or more materials selected from sesame oil, mineral oil, olive oil, petrolatum, water, ethanol, ethanol/water mixtures, isopropanol, isopropanol/water mixtures, dimethyl isosorbide, and mixtures thereof.

In another aspect, the present invention relates to a composition wherein the pharmaceutically acceptable carrier comprises sesame oil.

In another aspect, the present invention relates to a composition further comprising one or more ingredients selected from a penetration enhancer, a preservative, an antioxidant, an emulsifier, a surfactant, an emollient, a film forming agent, or a viscosity modifying agent, and mixtures thereof.

In another aspect, the present invention relates to a method for preparing a composition for topical delivery of tetrahydrocannabinol according to the present invention.

In another aspect, the present invention relates to a method for treating a condition involving or alternatively selected from pain, pruritus, muscle spasm, or inflammation comprising topically applying a therapeutically effective amount of tetrahydrocannabinol to a human patient in need thereof.

In another aspect, the present invention relates to a method for topically delivering a therapeutically effective amount of tetrahydrocannabinol to treat a condition selected from pain, pruritus, muscle spasm, or inflammation in a human patient in need thereof, comprising the step of: applying a composition comprising a therapeutically effective amount of tetrahydrocannabinol and a pharmaceutically acceptable carrier to the skin of said human patient.

In another aspect, the present invention relates to a method for treating pain or pruritus comprising topically applying a therapeutically effective amount of tetrahydrocannabinol to a human patient in need thereof.

In another aspect, the present invention relates to a method wherein said composition is applied at least once daily.

In another aspect, the present invention relates to a method wherein said composition is applied at least twice daily.

In another aspect, the present invention relates to a method wherein said composition is applied at least once weekly.

In another aspect, the present invention relates to a method wherein said composition is applied at least twice weekly.

In another aspect, the present invention relates to a method wherein said composition is applied at least once daily until the pain or pruritus is treated.

In another aspect, the present invention relates to a method for treating pain or pruritus comprising topically applying a composition of the present invention to a human patient in need thereof.

In another aspect, the present invention relates to a method for treating pain or pruritus comprising topically applying a therapeutically effective amount of tetrahydrocannabinol to a human patient in need thereof.

In another aspect, the present invention relates to a method wherein the pain is neuropathic or chronic inflammatory pain.

In another aspect, the present invention relates to a method wherein the pain is neuropathic pain.

In another aspect, the present invention relates to a method wherein the pain is chronic inflammatory pain.

In another aspect, the present invention relates to a method wherein the pruritus is neuropathic or chronic inflammatory pruritus.

In another aspect, the present invention relates to a method wherein the pruritus is neuropathic pruritus.

In another aspect, the present invention relates to a method wherein the pruritus is chronic inflammatory pruritus.

In another aspect, the present invention relates to the use of tetrahydrocannabinol in the manufacture of a medicament for topical delivery of a therapeutically effective amount of tetrahydrocannabinol for treating pain or pruritus in a human patient in need thereof.

These and other aspects of the present invention will become apparent from the disclosure herein.

### DEFINITIONS

As used herein, the following terms have the indicated meanings unless expressly stated to the contrary.

The term “neuropathic” as used herein with respect to pain or pruritus, means pain or pruritus associated with one or more nerves, including both peripheral and central nervous system nerves.

The term “pharmaceutically acceptable” is used herein with respect to the compositions, in other words the formulations, of the present invention. The pharmaceutical compositions of the present invention comprise a therapeutically effective amount of tetrahydrocannabinol and a pharmaceutically acceptable carrier. These carriers can contain a wide range of excipients. Pharmaceutically acceptable carriers are those conventionally known carriers having acceptable safety profiles. The compositions are made using common formulation techniques. See, for example, Remington's Pharmaceutical Sciences, 17th edition, edited by Alfonso R. Gennaro, Mack Publishing Company, Easton, Pa., 17th edition, 1985.

The term “subject” means a human patient or animal in need of treatment or intervention for pain or pruritus, particularly neuropathic or chronic inflammatory pain and/or pruritus.

The term “therapeutically effective” means an amount of tetrahydrocannabinol needed to provide a meaningful or demonstrable benefit, as understood by medical practitioners, to a subject, such as a human patient or animal, in need of treatment. Conditions, include for example pain or pruritus, particularly neuropathic or chronic inflammatory pain and/or pruritus, and other conditions such as muscle spasm or inflammation. For example, a meaningful or demonstrable benefit can be assessed or quantified using various clinical parameters. See, for pain: http://onlinelibrary.wiley.com/store/10.1002/acr.20543/asset/20543_ftp.pdf?v=1&t=j0b3 gt96&s=d528ae186ac91d091d1c56dea4cb6c0daff3c788, and for pruritus: http://www.itchforum.net/PDFs/3599.pdf. The demonstration of a benefit can also include those provided by models, including but not limited to in vitro models, in vivo models, and animal models. An example of such an animal model is the Freund's Adjuvant (Complete Freund's Adjuvant) Induced Model of Monoarthritis in Rat. Freund's adjuvant is a solution of an antigen (inactivated and dried mycobacteria) in an oil in water emulsion used to stimulate cell-mediated immunity.

The term “topical” as used herein with respect to pharmaceutical compositions means a composition that is applied to the skin or mucosal membrane of a subject, such as a human patient. A topical pharmaceutical composition is intended to have an effect at the site of application, i.e. in the tissue beneath the site of application, and does not result in significant drug concentrations in the blood and other tissues. Topical pharmaceutical compositions are in contrast to “transdermal” pharmaceutical compositions, which are absorbed through the skin or mucosal membranes and are intended to have a systemic effect in areas of the body away from the site of application. See, http://corporatepharmacy.ca/health-news/topical-vs-transdermal-meds, (2016).

Furthermore, the U.S. Food & Drug Administration has provided a standard for all routes of administration for drugs, i.e. “Route of Administration”. The following definitions are provided by the FDA for topical, transdermal, and transmucosal routes of drug administration.

| | | | | NCI* |
|---|---|---|---|---|
| | | SHORT | FDA | CONCEPT |
| NAME | DEFINITION | NAME | CODE | ID |
| TOPICAL | Administration to | TOPIC | 011 | C38304 |
| | a particular | | | |
| | spot on the | | | |
| | outer surface of | | | |
| | the body. | | | |
| TRANSDERMAL | Administration | T- | 358 | C38305 |
| | through the | DERMAL | | |
| | dermal layer of | | | |
| | the skin to the | | | |
| | systemic | | | |
| | circulation by | | | |
| | diffusion. | | | |
| TRANSMUCOSAL | Administration | T-MUCOS | 122 | C38283 |
| | across the | | | |
| | mucosa. | | | |

*National Cancer Institute

See, https://www.fda.gov/Drugs/DevelopmentApprovalProcess/FormsSubmissionRequirements/ ElectronicSubmissions/DataStandardsManualmonographs/ucm071667.htm.

The terms “treat,” “treating” or “treatment,” as used herein, include alleviating, abating or ameliorating the condition, e.g. pain or pruritus, or preventing or reducing the risk of contracting the condition or exhibiting the symptoms of the condition, ameliorating or preventing the underlying causes of the symptoms, inhibiting the condition, arresting the development of the condition, relieving the condition, causing regression of the condition, or stopping the symptoms of the condition, either prophylactically and/or therapeutically.

The methods of treatment using tetrahydrocannabinol or the pharmaceutical compositions of the present invention, in various embodiments also include the use of tetrahydrocannabinol in the manufacture of a medicament for the desired treatment, such as pain or pruritus.

### Tetrahydrocannabinol

The present invention utilizes a therapeutically effective amount of tetrahydrocannabinol and a pharmaceutically acceptable carrier for providing topical compositions for treating a condition involving or alternatively selected from pain, pruritus, muscle spasm, or inflammation.

Tetrahydrocannabinol, also known as THC, 1-trans-Δ⁹-tetrahydrocannabinol, delta-9-tetrahydrocannabinol, Marinol (as sold in capsule form by AbbVie Inc.) or dronabinol [by its International Nonproprietary Name (INN)], is believed to be the principal psychoactive constituent of Cannabis (or marijuana). Tetrahydrocannabinol is designated by CAS Registry No. 1972-08-03 and belongs to the chemical family known as cannabinoids. It has the full chemical name: 3-pentyl-6,6,9-trimethyl-6a,7,8.10a-tetrahydro-6H-dibenzo(b,d) pyran-1-ol and also corresponds to the IUPAC chemical name (−)-(6aR,10aR)-6,6,9-trimethyl-3-pentyl-6a,7,8,10a-tetrahydro-6H-benzo[c]chromen-1-ol.

Tetrahydrocannabinol can be a clear, amber or gold colored glassy solid when cold, which becomes viscous and sticky if warmed. Like most pharmacologically-active secondary metabolites of plants, tetrahydrocannabinol in the Cannabis plant is assumed to be involved in self-defense, perhaps against herbivores. Tetrahydrocannabinol also possesses high UVB absorption (280-31 nm), which, it has been speculated, could protect the plant from harmful UV radiation exposure. Tetrahydrocannabinol, along with its double bond isomers and their stereoisomers, is one of only three cannabinoids scheduled by the UN Convention on Psychotropic Substances (the other two are diemthylheptylpyran and parahexyl). It is currently classified as a Schedule I drug by the Drug Enforcement Agency. See, https://www.deadiversion.usdoj.gov/schedules/orangebook/c_cs_alpha.pdf. It is specifically still listed under Schedule I by US federal law under the Controlled Substances Act signed by the US Congress in 1970. Marijuana itself has now been legalized in several US states. See, Pate, David W. (1994). “Chemical ecology of Cannabis”. Journal of the International Hemp Association. 1 (29): 32-37; and Lydon, John; Teramura, Alan H. (1987). “Photochemical decomposition of cannabidiol in its resin base”. Phytochemistry. 26 (4): 1216-1217.

Tetrahydrocannabinol corresponds to the following chemical structure.

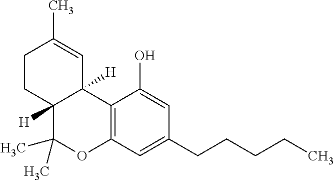 [SMILES: CCCCCc1cc(O)c2c(c1)OC(C)(C)[C@@H]1CCC(C)=C[C@@H]21]

Tetrahydrocannabinol has the chemical formula C₂₁H₃₀O₂ and a molar mass of 314.469 g/mol. It has a reported boiling point of 157° C. and a specific rotation of −152 degrees in ethanol, and a solubility in water of 0.0028 mg/ml at 20-23° C.

Tetrahydrocannabinol is available commercially. One such manufacturer is Cilag AG, a Swiss pharmaceutical company that is a subsidiary of Johnson & Johnson, and is supplied as a 15 to 25 percent solution in sesame oil. This material as supplied by Cilag can be further formulated or diluted, e.g. in additional sesame oil, to obtain a desired formulation for topical administration. For example, the 15 to 25 percent solution can be used as is, or further diluted down to 5 percent or 1 percent or any other desired concentration.

Preferably, the tetrahydrocannabinol has a purity of 95% by weight or greater as determined by HPLC. Compositions useful herein comprise about 0.005% to about 25% by weight tetrahydrocannabinol. Other useful compositions comprise from about 0.01% to about 15% by weight tetrahydrocannabinol, and from about 0.5% to about 10% by weight tetrahydrocannabinol. Examples of other useful compositions comprise about 0.1% by weight tetrahydrocannabinol, about 0.5% by weight tetrahydrocannabinol, about 1% by weight tetrahydrocannabinol, or about 5% by weight tetrahydrocannabinol.

When in the form of a unit dosage for administration to a human patient, the following human dosages are useful: a unit dosage composition comprising about 0.001 to about 1 mg/kg of tetrahydrocannabinol, based on the weight of the human patient and a unit dosage composition comprising about 0.003 to about 0.5 mg/kg of tetrahydrocannabinol, based on the weight of the human patient. Examples of other useful unit dosages comprise about 0.003 mg/kg of tetrahydrocannabinol, based on the weight of the human patient, or about 0.01 mg/kg of tetrahydrocannabinol, based on the weight of the human patient, or about 0.05 mg/kg of tetrahydrocannabinol, based on the weight of the human patient, or about 0.1 mg/kg of tetrahydrocannabinol, based on the weight of the human patient, or about 0.5 mg/kg of tetrahydrocannabinol based on the weight of the human patient.

Furthermore, because the present invention is related to topical compositions and because it is highly desirable to limit systemic exposure, the unit dosage should demonstrate at least one of the following pharmacokinetic parameters selected from a Cₘₐₓ less than about 1.32 ng/ml or an AUC less than about 2.88 ng hr/ml.

### Formulations for Topical Administration

The formulations of the present invention can be made using standard formulation and mixing techniques familiar to one of ordinary skill in the art of pharmaceuticals and formulations.

In one aspect, the present invention comprises a pharmaceutically effective amount of tetrahydrocannabinol and a pharmaceutically acceptable carrier.

Useful herein are compositions wherein the pharmaceutically acceptable carrier is selected from one or more materials selected from sesame oil, mineral oil, olive oil, petrolatum, water, ethanol, ethanol/water mixtures, isopropanol, isopropanol/water mixtures, and dimethyl isosorbide. Other examples of pharmaceutical carriers include those selected from oils derived from fruits or vegetables or flowers or nuts or seeds (including but not limited to sesame oil, peanut oil, and castor oil), alcohols (including but not limited to ethanol, benzyl alcohol, and isopropyl alcohol), dipropylene glycol, ethyl acetate, ethyl lactate, ethyl oleate, glycerin, isopropyl myristate, isopropyl palmitate, medium-chain triglycerides, mineral oil, polyethylene glycol, propylene glycol, tricaprylin, and water. A specific example of a pharmaceutically acceptable carrier is sesame oil.

Various additional ingredients can be used in the compositions of the present invention. In some, but not all, embodiments, the compositions can comprise one or more further ingredients selected from a penetration enhancer, a preservative, an antioxidant, an emulsifier, an emollient, or a viscosity modifying agent.

In one aspect, a penetration enhancer can be included. In another aspect, a preservative can be included. In another aspect, an antioxidant can be included. In another aspect, a viscosity modifying agent can be included. In another aspect, a surfactant or wetting agent can be included. In another aspect, a film forming agent can be included. In another aspect, an emulsifier can be included. In another aspect, an emollient can be included. In another aspect, the pharmaceutical composition is in the form selected from the group consisting of a gel, ointment, lotion, emulsion, cream, foam, mousse, liquid, paste, jelly, tape, spray, suspension, dispersion, aerosol, or film forming agent.

In another aspect, the pharmaceutically acceptable carrier can comprise a material selected from the group consisting of alcohols (including but not limited to ethanol, benzyl alcohol, or isopropyl alcohol), acetone, albumin, oils derived from fruits or vegetables or flowers or nuts or seeds (including but not limited to almond oil, corn oil, cottonseed oil, coconut oil, sesame oil, olive oil, peanut oil, safflower oil, soybean oil, or sunflower oil), benzyl benzoate, butylene glycol, carbon dioxide, castor oil, dibutyl phthalate, diethyl phthalate, diethylene glycol, diethylene glycol monoethyl ether, dimethyl ether, dimethyl phthalate, dimethyl sulfoxide, dimethylacetamide, dipropylene glycol, ethyl acetate, ethyl lactate, ethyl oleate, glycerin, glyceryl monostearate, glycofurol, isopropyl myristate, isopropyl palmitate, light mineral oil, mineral oil, medium-chain triglycerides, methyl lactate, monoethanolamine, octyldodecanol, polyethylene glycol, polyoxyl 35 castor oil, propylene carbonate, propylene glycol, pyrrolidone, triacetin, tricaprylin, triethanolamine, triethyl citrate, triolein, and water, or a combination thereof. These components can be employed and used at levels appropriate for the formulation based on the knowledge of one with ordinary skill in the pharmaceutical and formulation arts. The amounts could range from under 1 percent by weight to up to 90 percent or even over 99 percent by weight.

In another aspect, the at least one penetration enhancer can be selected from the group consisting of alcohols (including but not limited to ethanol, benzyl alcohol, oleyl alcohol, or isopropyl alcohol), diethyl sebacate, diethylene glycol, dimethyl sulfoxide, glyceryl monooleate, glycofurol, isopropyl myristate, isopropyl palmitate, light mineral oil, lauric acid, linoleic acid, menthol, myristic acid, oleic acid, palmitic acid, polyoxyethylene alkyl ethers, polyoxyglycerides, propylene glycol, propylene glycol monolaurate, pyrrolidone, sodium lauryl sulfate, squalane, thymol, tricaprylin, triolein, and transcutol, or a combination thereof. These components can be employed and used at levels appropriate for the formulation based on the knowledge of one with ordinary skill in the pharmaceutical and formulation arts. The amounts could range from under 1 percent by weight to up to 90 percent by weight.

In another aspect, the at least one preservative can be selected from the group consisting of parabens (including butylparabens, ethylparabens, methylparabens, and propylparabens), acetone sodium bisulfite, alcohol, benzalkonium chloride, benzethonium chloride, benzoic acid, benzyl alcohol, boric acid, bronopol, butylated hydroxyanisole, butylene glycol, calcium acetate, calcium chloride, calcium lactate, cetrimide, cetylpyridinium chloride, chlorhexidine, chlorobutanol, chlorocresol, chloroxylenol, cresol, edetic acid, glycerin, hexetidine, imidurea, isopropyl alcohol, monothioglycerol, pentetic acid, phenol, phenoxyethanol, phenylethyl alcohol, phenylmercuric acetate, phenylmercuric borate, phenylmercuric nitrate, potassium benzoate, potassium metabisulfite, potassium nitrate, potassium sorbate, propionic acid, propyl gallate, propylene glycol, propylparaben sodium, sodium acetate, sodium benzoate, sodium borate, sodium lactate, sodium metabisulfite, sodium propionate, sodium sulfite, sorbic acid, sulfur dioxide, thimerosal, zinc oxide and N-acetylcysteine, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one antioxidant can be selected from the group consisting of acetone sodium bisulfite, alpha tocopherol, ascorbic acid, ascorbyl palmitate, butylated hydroxyanisole, butylated hydroxytoluene, citric acid monohydrate, dodecyl gallate, erythorbic acid, fumaric acid, malic acid, mannitol, sorbitol, monothioglycerol, octyl gallate, potassium metabisulfite, propionic acid, propyl gallate, sodium ascorbate, sodium formaldehyde sulfoxylate, sodium metabisulfite, sodium sulfite, sodium thiosulfate, sulfur dioxide, thymol, vitamin E polyethylene glycol succinate, and N-acetylcysteine, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one emulsifier can be selected from the group consisting of acacia, agar, ammonium alginate, calcium alginate, carbomer, carboxymethylcellulose sodium, cetostearyl alcohol, cetyl alcohol, cholesterol, diethanolamine, glyceryl monooleate, glyceryl monostearate, hectorite, hydroxypropyl cellulose, hydroxypropyl starch, hypromellose, lanolin, lanolin alcohols, lauric acid, lecithin, linoleic acid, magnesium oxide, medium-chain triglycerides, methylcellulose, mineral oil, monoethanolamine, myristic acid, octyldodecanol, oleic acid, oleyl alcohol, palm oil, palmitic acid, pectin, phospholipids, poloxamer, polycarbophil, polyoxyethylene alkyl esthers, polyoxyethylene castor oil derivatives, polyoxyethylene sorbitan fatty acid esters, polyoxyethylene stearates, polyoxyl 15 hydroxystearate, polyoxylglycerides, potassium alginate, propylene glycol alginate, propylene glycol dilaurate, propylene glycol monolaurate, saponite, sodium borate, sodium citrate dehydrate, sodium lactate, sodium lauryl sulfate, sodium stearate, sorbitan esters, starch, stearic acid, sucrose stearate, tragacanth, triethanolamine, tromethamine, vitamin E polyethylene glycol succinate, wax, and xanthan gum, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one emollient can be selected from the group consisting of almond oil, aluminum monostearate, butyl stearate, canola oil, castor oil, cetostearyl alcohol, cetyl alcohol, cetyl palmitate, cholesterol, coconut oil, cyclomethicone, decyl oleate, diethyl sebacate, dimethicone, ethylene glycol stearates, glycerin, glyceryl monooleate, glyceryl monostearate, isopropyl isostearate, isopropyl myristate, isopropyl palmitate, lanolin, lanolin alcohols, lecithin, mineral oil, myristyl alcohol, octyldodecanol, oleyl alcohol, palm kernel oil, palm oil, petrolatum, polyoxyethylene sorbitan fatty acid esters, propylene glycol dilaurate, propylene glycol monolaurate, safflower oil, squalene, sunflower oil, tricaprylin, triolein, wax, xylitol, and zinc acetate, or a combination thereof. These components can be employed and used at levels appropriate for the formulation based on the knowledge of one with ordinary skill in the pharmaceutical and formulation arts. The amounts could range from under 1 percent by weight to up to 60 percent by weight.

In another aspect, the at least one viscosity modifying agent can be selected from the group consisting of acacia, agar, alginic acid, aluminum monostearate, ammonium alginate, attapulgite, bentonite, calcium alginate, calcium lactate, carbomer, carboxymethylcellulose calcium, carboxymethylcellulose sodium, carrageenan, cellulose, ceratonia, ceresin, cetostearyl alcohol, cetyl palmitate, chitosan, colloidal silicon dioxide, corn syrup solids, cyclomethicone, ethylcellulose, gelatin, glyceryl behenate, guar gum, hectorite, hydrophobic colloidal silica, hydroxyethyl cellulose, hydroxyethylmethyl cellulose, hydroxypropyl cellulose, hydroxypropyl starch, hypromellose, magnesium aluminum silicate, maltodextrin, methylcellulose, myristyl alcohol, octyldodecanol, palm oil, pectin, polycarbophil, polydextrose, polyethylene oxide, polyoxyethylene alkyl ethers, polyvinyl alcohol, potassium alginate, propylene glycol alginate, pullulan, saponite, sodium alginate, starch, sucrose, sugar, sulfobutylether β-cyclodextrin, tragacanth, trehalose, and xanthan gum, or a combination thereof. These components can be employed and used at levels appropriate for the formulation based on the knowledge of one with ordinary skill in the pharmaceutical and formulation arts. The amounts could range from under 1 percent by weight to up to 90 percent or even over 99 percent by weight.

In another aspect, the at least one film forming agent can be selected from the group consisting of ammonium alginate, chitosan, colophony, copovidone, ethylene glycol and vinyl alcohol grafted copolymer, gelatin, hydroxypropyl cellulose, hypromellose, hypromellose acetate succinate, polymethacrylates, poly(methyl vinyl ether/maleic anhydride), polyvinyl acetate dispersion, polyvinyl acetate phthalate, polyvinyl alcohol, povidone, pullulan, pyroxylin, and shellac, or a combination thereof. These components can be employed and used at levels appropriate for the formulation based on the knowledge of one with ordinary skill in the pharmaceutical and formulation arts. The amounts could range from under 1 percent by weight to up to 90 percent or even over 99 percent by weight.

In another aspect, the at least one surfactant or wetting agent can be selected from the group consisting of docusate sodium, phospholipids, sodium lauryl sulfate, benzalkonium chloride, cetrimide, cetylpyridinium chloride, alpha tocopherol, glyceryl monooleate, myristyl alcohol, poloxamer, polyoxyethylene alkyl ethers, polyoxyethylene castor oil derivatives, polyoxyethylene sorbitan fatty acid esters, polyoxyethylene stearates, polyoxyl 15 hydroxystearate, polyoxyglycerides, propylene glycol dilaurate, propylene glycol monolaurate, sorbitan esters, sucrose stearate, tricaprylin, and vitamin E polyethylene glycol succinate, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, a buffering agent can be included. In another aspect, an emollient can be included. In another aspect, an emulsifying agent can be included. In another aspect, an emulsion stabilizing agent can be included. In another aspect, a gelling agent can be included. In another aspect, a humectant can be included. In another aspect, an ointment base or oleaginous vehicle can be included. In another aspect, a suspending agent can be included.

One of ordinary skill in the pharmaceutical and formulation arts can determine the appropriate levels of the essential and optional components of the compositions of the present invention.

Methods of preparing the tetrahydrocannabinol compositions are also intended as part of the present invention and would be apparent to one of ordinary skill in the pharmaceutical and formulation arts using standard formulation and mixing techniques.

### Methods of Treatment

The present invention utilizes a therapeutically effective amount of tetrahydrocannabinol and a pharmaceutically acceptable carrier for providing topical compositions for treating a condition involving or alternatively selected from pain, pruritus, muscle spasm, or inflammation in a human patient or animal in need thereof. More specific conditions are, for example, pain and pruritus, particularly neuropathic or chronic inflammatory pain and/or pruritus.

The methods comprise topically applying a therapeutically effective amount of tetrahydrocannabinol to the human patient or animal in need thereof. When a human patient is being treated, the composition is applied to the skin of said human.

Various dosing regimens can be prescribed and used based on the skill and knowledge of the physician or other practitioner. In some embodiments, a unit dosage of the composition, as described herein can be applied at least once daily. In other embodiments, a unit dosage of the composition can be applied at least twice daily, or at least once weekly, or at least twice weekly.

Topical administration of the composition can be continued in the judgment of the physician or practitioner until the desired therapeutic benefit is achieved, i.e. until the pain or pruritus is treated. In some instances, it can be desirable to continue long term or chronic therapy.

Because the tetrahydrocannabinol is relatively lipophilic, a dosing regimen can be designed to provide an initial loading or depot effect to the underlying tissue at a relatively high dose and/or frequent treatment regime, wherein the dose and/or the dosing frequency is subsequently decreased to maintain the desired therapeutic effect. For example, it can in some instances be useful to dose twice daily for up to a week at a high topical dose of 0.2 mg/kg, and then decrease down to a once daily dose of 0.02 mg/kg. Other dosage amounts and regimens can be designed based upon the judgment of the physician or practitioner.

### EXAMPLES

The following examples further describe and demonstrate embodiments within the scope of the present invention. The Examples are given solely for purpose of illustration and are not to be construed as limitations of the present invention, as many variations thereof are possible without departing from the spirit and scope of the invention.

### Example 1: Preparation of a Composition for Topical Delivery

Tetrahydrocannabinol (dronabinol) was purchased from Cilag AG as a 15 to 25 percent solution in sesame oil. This material corresponded to 158.4 g of tetrahydrocannabinol as active ingredient or Active Pharmaceutical Ingredient (API) in sesame oil per 0.900 kilograms total formulation. The material was further diluted using standard formulation techniques to obtain formulations having approximately 15 percent, 5 percent, and 1 percent tetrahydrocannabinol. The originally purchased sesame oil solution and the diluted material was generally stored between 2 to 8° C. until ready for administration.

This tetrahydrocannabinol composition is useful for topical administration to a human patient or animal for the treatment of conditions such as pain or pruritus.

### Example 2: Evaluation of a Topical Composition in an Arthritis Model

The therapeutic efficacy of tetrahydrocannabinol as a topical treatment in the Complete Freund's Adjuvant (CFA) induced model of monoarthritis in rats was evaluated in this study. The study was conducted against morphine as a positive control and also a vehicle control. The dose and route of administration from the positive control group was employed to ensure immediate and maximal analgesia against which the tetrahydrocannabinol groups could be compared.

Forty-five male Sprague Dawley® (SD) rats were injected into the tibio-tarsal joint intra-articular using 27 gauge needles, with CFA to generate the neuropathic pain model. See, Butler, S. H. et al., “A limited arthritic model for chronic pain studies in the rat”, Pain, 1992 January:48(1):73-81 and J. Bennett, Gary. (2006). Can We Distinguish Between Inflammatory and Neuropathic Pain?. Pain Research and Management. 11. 11A-15A. CFA is a solution of an antigen (inactivated and dried mycobacteria) in an oil-in-water emulsion used to stimulate cell-mediated immunity. Two weeks following the injection, the rats were allocated to the various groups randomly and in accordance with their Von Frey and Incapacitance test results (i.e. standard tests relating to skin pain and sensitivity). See, https://www.ncbi.nlm.nih.gov/pmc/articles/PMC4110928/ and https://www.ncbi.nlm.nih.gov/pmc/articles/PMC3950312/. The animals were then treated topically once a day with control vehicle (sesame oil) or tetrahydrocannabinol in sesame oil at concentrations of 1, 5 or 15% topically for five (5) consecutive days. Approximately 100 μl was dispersed on to the area around the CFA injection site and spread on the site by gentle massaging with a gloved hand. The area was wrapped with a Petflex® No Chew Bandage to avoid oral consumption of the test compositions. The bandages were removed prior to the Von Frey and Incapacitance tests. Morphine was used as a positive control. The morphine control was diluted from a commercial preparation and administered intraperitoneally (IP) as an aqueous solution at a dosage of 10 mg/kg morphine up to one hour before testing on the days the Von Frey and Incapacitance tests (Days 16 and 19) were conducted. The effect of the various test compositions on pain relief, as indicated by the Von Frey and Incapacitance tests, was evaluated.

### Body Weight

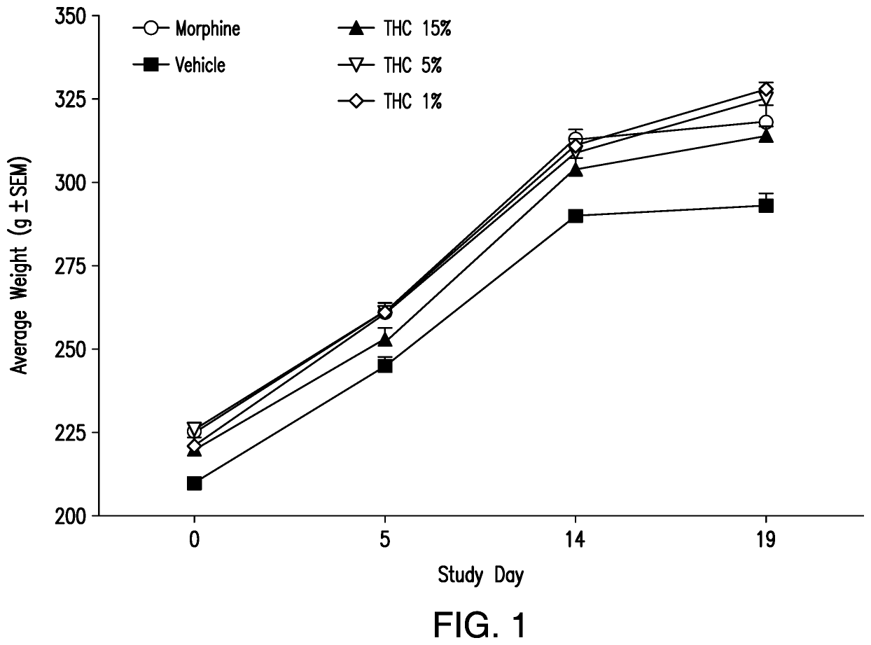

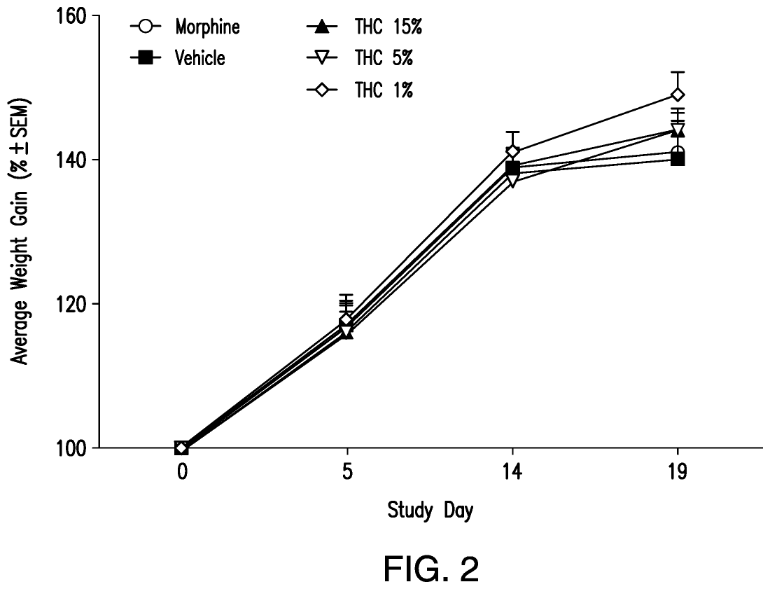

All rats in the five study groups exhibited weight increases throughout the study, leveling off on Day 19. This positive body weight increase could indicate that the treatment was well tolerated. When measured in grams, the animals in the vehicle group gained significantly less weight compared to the other treated groups (see FIG. 1). However, this was not statistically significant when the percentage of body weight from day zero was calculated (see FIG. 2). No statistically significant difference was found between the morphine group and the three THC treated groups (see Table 1, below) indicating they have a similar effect on the body weight gain (grams).

TABLE 1
Statistical analysis of Body
Weight (g) throughout the study.

| | Treatment Group | | | |
|---|---|---|---|---|
| Study Day | Morphine | THC 15% | THC 5% | THC 1% |
| Day 5 | *** | — | *** | *** |
| Day 14 | **** | ** | **** | **** |
| Day 19 | **** | **** | **** | **** |

** p < 0.01 compared to vehicle treatment.
*** p < 0.001 compared to vehicle treatment.
**** p < 0.0001 compared to vehicle treatment.

According to the above analysis of the body weights, all tetrahydrocannabinol solutions (15%, 5% and 1%) provided the same potential increase in the rats' appetite that was comparable to the effect of the morphine treatment. This trend was also visible (although not statistically significant) in the analysis of the percent of body weight gain on day 19 as a calculated percentage from day 0, where all the tetrahydrocannabinol groups had a slightly higher weight gain on day 19 than the control group.

Incapacitance (Weight Bearing Test): Weight bearing changes in the rats with monoarthritis were monitored using an incapacitance testing device. Each rat was placed so that each hind paw rested on a separate force plate on the incapacitance apparatus, and the weight borne by each hind limb was measured for five (5) seconds. The ratio of the weight borne by the right to left hind limb was calculated. The mean of three consecutive measurements for each rat was recorded. Weight bearing function (Incapacitance Test) was performed on all of the animals at baseline (Day 14), Day 16, and Day 19 for a total of three times (nine animals/group/time point).

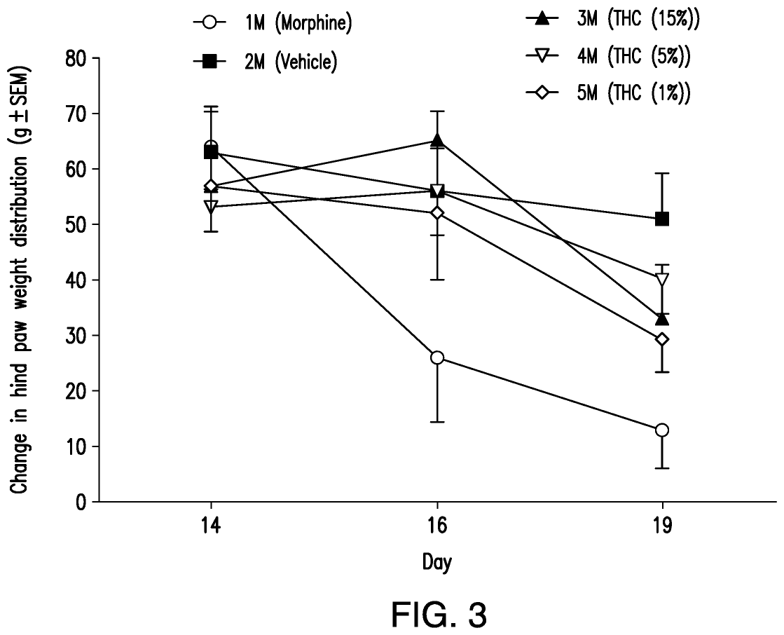

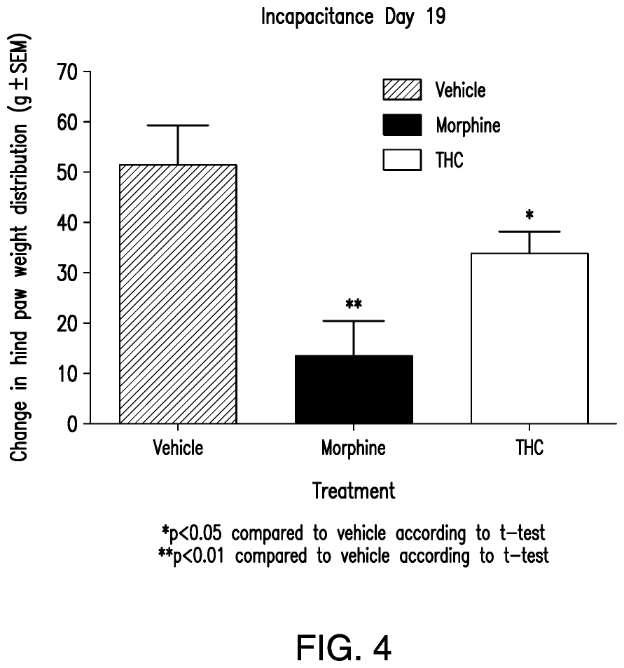

As seen in FIG. 3, the morphine positive control exhibited a significant effect on pain perception of the animals, compared to the vehicle control. However, a trend revealing tetrahydrocannabinol activity was observed on Day 19. Furthermore, no clear-cut dose-response was observed, as it seemed that even the lowest dose of 1% concentration reached a plateau level of activity. Thus, combining all the tetrahydrocannabinol groups into one group was scientifically justified as seen in FIG. 4. After combining all the tetrahydrocannabinol groups together, the trend of a positive effect on pain reduction became statistically significant as shown in FIG. 4. Analyzing the results according to “percent responders” (animals in which pain reduction was >50%), revealed a similar pattern as shown in Table 2. Combining all the tetrahydrocannabinol treated rats showed lower effects compared to morphine, but clearly higher than vehicle.

TABLE 2
Percent of animals presenting
above 50% reduction in pain perception.

| Treatment Group | Vehicle | Morphine | THC |
|---|---|---|---|
| Percent | 11% (1/9) | 66% (6/9) | 24% (7/27) |
| Responders | | | |

Tactile Allodynia (von Frey test): The von Frey test consists of thin calibrated plastic filaments that are applied to the plantar surface of the hindpaw. Von Frey filaments of different gauges or stiffness are used to determine the threshold that elicits a hindpaw withdrawal response. The mechanical withdrawal threshold is defined as the minimum gauge von Frey filament that elicits a withdrawal reflex. See, www.iacuc.ucsf.edu/policies/mechanicalsensitivity.doc.

The rats were placed inside a Plexiglas chamber for a 10-15 minute acclimation period. Subsequently the rats were evaluated for tactile allodynia, i.e. pain sensitization and nociception, using a von Frey Filament (VFF) ranging from the thinnest 0.6 g filament up to the thickest 15 g (0.6, 1.4, 2, 4, 6, 8, 10, 15 g) in the following manner: the technician approached the animal from below using the thinnest von Frey filament and touched the hind paw 5 consecutive times, or until the rat responded. If no response occurred, testing proceeded to the next ascending filament. Once a withdrawal response was established, the paw was retested, with the preceding descending filament until no response was observed. The lag time between filaments, ascending or descending was approximately 90 seconds. Each animal had both hind paws tested in this manner (first the injected leg and then the control leg). The lowest amount of force required to elicit a response was recorded as withdrawal threshold in grams. The Von-Frey test was performed at baseline (Day 14), Day 16 and Day 19 for a total of 3 times (nine animals/group/time point).

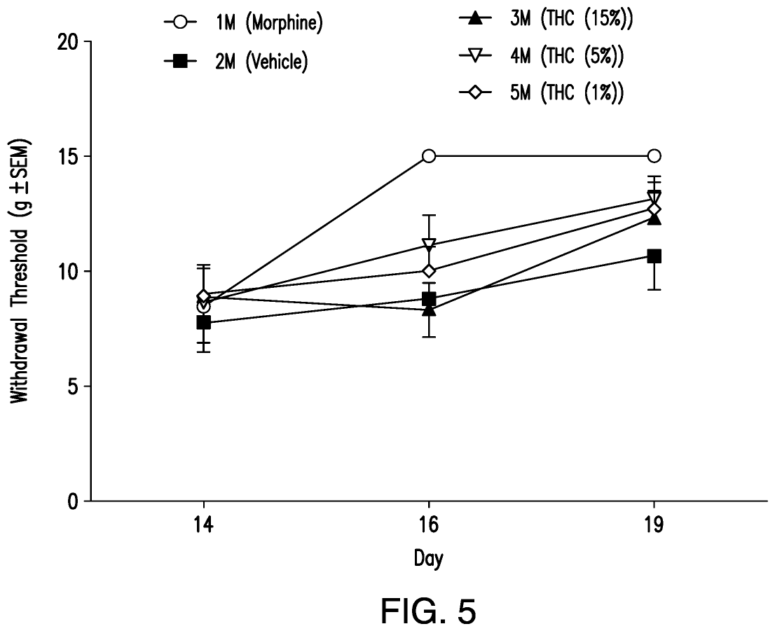

The von Frey test results exhibited a similar outcome as found for the Incapacitance test (see FIG. 5). Morphine exhibited a considerable effect on pain reduction while the tetrahydrocannabinol solutions revealed a similar trend of efficacy with no dose-response relationship.

In summary, all animals in all the treatment groups gained weight throughout the study, indicating the treatments were well tolerated. The positive control of 10 mg/kg of morphine injected IP exhibited significant pain reducing effect in both tests on both tested days (16 and 19). This morphine dose in rats is approximately one order of magnitude higher than the commonly used equivalent dose in humans for clinical purposes. See, Tveita T et al., “A controlled comparison between single doses of intravenous and intramuscular morphine with respect to analgesic effects and patient safety”, Acta Anaesthesiol Scand. 2008; 52:920-925. The tetrahydrocannabinol examined tested groups showed an accumulative effect, rather than an immediate effect as seen in the morphine treated group, which therefore was observable only on Day 19. All three tetrahydrocannabinol dosage concentrations revealed a similar trend of efficacy, with no clear-cut dose response relationship, indicating that a positive plateau effect had been achieved at the lowest dose. When the data from the three tetrahydrocannabinol groups were combined, this combined group reached statistical significance compared to the vehicle treatment for the Incapacitance test on Day 19.

These results suggest the potential of pain and pruritus amelioration by the topical administration of tetrahydrocannabinol.

### INCORPORATION BY REFERENCE

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the methods and systems of the present invention, where the term comprises is used with respect to the recited steps of the methods or components of the compositions, it is also contemplated that the methods and compositions consist essentially of, or consist of, the recited steps or components. Furthermore, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

Furthermore, it should be recognized that in certain instances a composition can be described as being composed of the components prior to mixing, because upon mixing certain components can further react or be transformed into additional materials.

All percentages and ratios used herein, unless otherwise indicated, are by weight.

1. A composition for topical delivery of tetrahydrocannabinol comprising a therapeutically effective amount of tetrahydrocannabinol and a pharmaceutically acceptable carrier.

2. A composition according to claim 1 wherein the tetrahydrocannabinol has a purity of 95% by weight or greater as determined by HPLC.

3. A composition according to claim 1 comprising about 0.005% to about 25% by weight tetrahydrocannabinol.

4-11. (canceled)

12. A composition according to claim 1 for administration to a human patient or animal.

13. A composition according to claim 12 for administration to a human patient.

14. A composition according to claim 13 in the form of a unit dosage composition.

15. A unit dosage composition according to claim 14 comprising about 0.001 to about 1 mg/kg of tetrahydrocannabinol, based on the weight of the human patient.

16.-21. (canceled)

22. A unit dosage composition according to claim 14 demonstrating at least one of the following pharmacokinetic parameters selected from a Cₘₐₓ less than about 1.32 ng/ml or an AUC less than about 2.88 ng hr/ml.

23. A composition according to claim 1 wherein the pharmaceutically acceptable carrier comprises one or more materials selected from plant-based oils, alcohols, dipropylene glycol, ethyl acetate, ethyl lactate, ethyl oleate, glycerin, isopropyl myristate, isopropyl palmitate, medium-chain triglycerides, mineral oil, petrolatum, silicone oil polyethylene glycol, propylene glycol, tricaprylin, dimethyl isosorbide, water, and mixtures thereof.

24. A composition according to claim 23 wherein the plant based oil is selected from oils derived from fruits, vegetables, flowers, nuts, or seeds.

25. A composition according to claim 23 wherein the plant based oil is selected from sesame oil, olive oil, peanut oil, castor oil, almond oil, canola oil, corn oil, cottonseed oil, safflower oil, soybean oil, sunflower oil, and mixtures thereof.

26. A composition according to claim 23 wherein the alcohol is selected from ethanol, benzyl alcohol, isopropyl alcohol, and mixtures thereof.

27.-28. (canceled)

29. A composition according to claim 1 further comprising one or more ingredients selected from a penetration enhancer, a preservative, an antioxidant, an emulsifier, a surfactant, an emollient, a film forming agent, or a viscosity modifying agent, and mixtures thereof.

30. (canceled)

31. A method for treating a condition involving or alternatively selected from pain, pruritus, muscle spasm, or inflammation comprising topically applying a therapeutically effective amount of tetrahydrocannabinol to a human patient in need thereof.

32. A method for topically delivering a therapeutically effective amount of tetrahydrocannabinol to treat a condition selected from pain, pruritus, muscle spasm, or inflammation in a human patient in need thereof, comprising the step of:
applying a composition comprising a therapeutically effective amount of tetrahydrocannabinol and a pharmaceutically acceptable carrier to the skin of said human patient.

33. A method for treating pain or pruritus comprising topically applying a therapeutically effective amount of tetrahydrocannabinol to a human patient in need thereof.

34.-37. (canceled)

38. A method according to claim 33 wherein said composition is applied at least once daily until the pain or pruritus is treated.

39.-41. (canceled)

42. A method according to claim 31 wherein the pain is neuropathic pain.

43. A method according to claim 31 wherein the pain is chronic inflammatory pain.

44. (canceled)

45. A method according to claim 31 wherein the pruritus is neuropathic pruritus.

46. A method according to claim 31 wherein the pruritus is chronic inflammatory pruritus.

47. (canceled)
